(19) United States
(12) Patent Application Publication
(10) Pub. No.: US 2015/0094580 A1
(43) Pub. Date: Apr. 2, 2015

(54) ULTRASONIC DIAGNOSTIC DEVICE AND LOCUS DISPLAY METHOD

(71) Applicant: HITACHI ALOKA MEDICAL, LTD., Mitaka-shi, Tokyo (JP)

(72) Inventor: Koji Waki, Mitaka-shi (JP)

(21) Appl. No.: 14/390,700

(22) PCT Filed: Feb. 21, 2013
(86) PCT No.: PCT/JP2013/054306

§ 371 Date: Oct. 3, 2014

(30) Foreign Application Priority Data
Apr. 13, 2012 (JP) 2012-092259

(51) Int. Cl. A61B 8/00; A61B 8/08; A61B 8/15

(52) U.S. Cl. CPC A61B 8/54; A61B 8/15; A61B 8/485; A61B 8/463
USPC 600/438; 382/131

(57) ABSTRACT

In an ultrasonic diagnostic device, on the basis of a displacement distribution in a 2D direction, a locus related to displacement in a discretionary region of an ultrasonic image is formed. The ultrasonic diagnostic device includes: an image forming unit (tomographic image forming unit and elastic image forming unit) for forming an ultrasonic image of a diagnosis location on a subject via an ultrasonic probe; an image display for displaying the ultrasonic image; and a locus forming unit (display parameter calculation unit, display data storing unit, 2D locus creating unit) that, on the basis of a displacement distribution in a 2D direction in a discretionary region of the ultrasonic image, forms a locus related to displacement in such region, and that display the formed locus on the image display.

### TECHNICAL FIELD

The present invention relates to an ultrasound diagnostic apparatus that displays an ultrasound image of the inside of a body of a subject using an ultrasound and supplies the image for diagnosis, and to a trajectory display method.

### BACKGROUND ART

An ultrasound diagnostic apparatus transmits an ultrasound toward an inside of a subject using an ultrasound probe, receives a reflection echo signal of the ultrasound corresponding to the structure of the living body tissue from the inside of the subject, forms an ultrasound image of the inside of the subject body, and displays the image for diagnosis (refer to Patent Documents 1 and 2).

A technique is known in which a function to calculate a time sequential similarity of a two-dimensional or three-dimensional local region, so-called pattern matching function, is provided as one application function of the ultrasound diagnosis apparatus, and a tissue such as a cardiac muscle is tracked. For example, Patent Document 1 describes that periodicity of motion is linked to diagnostic information based on correlation between a blood vessel diameter obtained by the tracking process and a change rate thereof. Patent Document 2 proposes setting an appropriate search range of the pattern matching, to check regularity of the motion.

### RELATED ART REFERENCES

### Patent Documents

### [Patent Document 1] JP 2002-17728 A

### [Patent Document 2] Japanese Patent No. 4659974

### DISCLOSURE OF INVENTION

### Technical Problem

However, the tracking techniques described in Patent Documents 1 and 2 relate to an amount of displacement of a local measurement point in a blood vessel wall or a cardiac muscle, and employ methods using displacement data along a direction of calculation of elasticity. For example, in regions of a mammary gland and a liver, displacements in two-dimensional directions, vertical and horizontal, may be irregularly generated within the region. Therefore, the tracking techniques at the measurement points are not suited for diagnosis of a region of a wide range.

An advantage of the present invention is that, in an ultrasound diagnostic apparatus, a trajectory related to displacements in two-dimensional directions in an arbitrary region of a subject is formed.

### Solution to Problem

In order to achieve the advantage described above, according to one aspect of the present invention, there is provided an ultrasound diagnostic apparatus comprising: an image forming unit that forms an ultrasound image of a diagnosis site of a subject through an ultrasound probe; an image display that displays the ultrasound image; and a trajectory forming unit that forms, based on a displacement distribution in two-dimensional directions in an arbitrary region of the ultrasound image, a trajectory related to a displacement of the region, and that causes the trajectory to be displayed on the image display.

According to another aspect of the present invention, there is provided a method of displaying a trajectory, comprising the steps of: forming an ultrasound image of a diagnosis site of a subject through an ultrasound probe; forming, based on a displacement distribution in two-dimensional directions in an arbitrary region of the ultrasound image, a trajectory related to a displacement of the region; and displaying the ultrasound image and the trajectory.

### Advantageous Effect

According to various aspects of the present invention, a trajectory related to a displacement in two-dimensional directions in an arbitrary region of a subject can be formed.

### BRIEF DESCRIPTION OF DRAWINGS

FIG. 1 is a block diagram exemplifying an ultrasound diagnostic apparatus according to a first preferred embodiment of the present invention.

FIG. 2 is a block diagram exemplifying a structure of a trajectory forming unit according to the first preferred embodiment of the present invention.

FIG. 3 is a diagram exemplifying displaying of an image on an image display according to the first preferred embodiment of the present invention.

FIG. 4 is a diagram exemplifying a trajectory (two-dimensional displacement coordinates) including a rectangular guide in a second preferred embodiment of the present invention.

FIG. 5 is a diagram exemplifying a trajectory (two-dimensional displacement coordinates) including a circular guide in the second preferred embodiment of the present invention.

FIG. 6 is a diagram exemplifying a trajectory (two-dimensional displacement coordinates) including a circular guide and with a narrower appropriate range than the guide shown in FIG. 5, in the second preferred embodiment of the present invention.

FIG. 7 is a diagram exemplifying a trajectory (displacement histogram) in a third preferred embodiment of the present invention.

FIG. 8 is a diagram exemplifying displaying of an image on an image display according to a fourth preferred embodiment of the present invention.

FIG. 9 is a diagram exemplifying displaying of an image on an image display according to a fifth preferred embodiment of the present invention.

FIG. 10 is a schematic diagram exemplifying a displacement detection method in a displacement measurement unit when a two-dimensional displacement image is formed in the fifth preferred embodiment of the present invention.

FIG. 11 is a diagram exemplifying a state of displacement detection of an organ displaced in a direction inclined with a predetermined angle with respect to an ultrasound scanning direction in a sixth preferred embodiment of the present invention.

FIG. 12 is a diagram exemplifying a trajectory (two-dimensional displacement coordinates) in a parameter acquisition region which is set for an organ shown in FIG. 11, according to the sixth preferred embodiment of the present invention.

FIG. 13 is a diagram exemplifying two-dimensional displacement coordinates, with a displacement direction angle θ calculated, in the sixth preferred embodiment of the present invention.

FIG. 14 is a diagram exemplifying a state of displacement detection of an organ shown in FIG. 11 by inclining the ultrasound scanning direction by a displacement direction angle θ in the sixth preferred embodiment of the present invention.

FIG. 15 is a diagram exemplifying a trajectory (two-dimensional displacement coordinates in the parameter acquisition region which is set for the organ shown in FIG. 11) formed by inclining the ultrasound scanning direction by a displacement direction angle θ in the sixth preferred embodiment of the present invention.

FIG. 16 is a diagram exemplifying a guide in a seventh preferred embodiment of the present invention.

FIG. 17 is a diagram exemplifying a message in the seventh preferred embodiment of the present invention.

FIG. 18 is a diagram exemplifying displaying of an image on an image display in an eighth preferred embodiment of the present invention.

FIG. 19 is a block diagram exemplifying a structure of a trajectory forming unit according to the eighth preferred embodiment of the present invention.

### BEST MODE FOR CARRYING OUT THE INVENTION

### First Preferred Embodiment

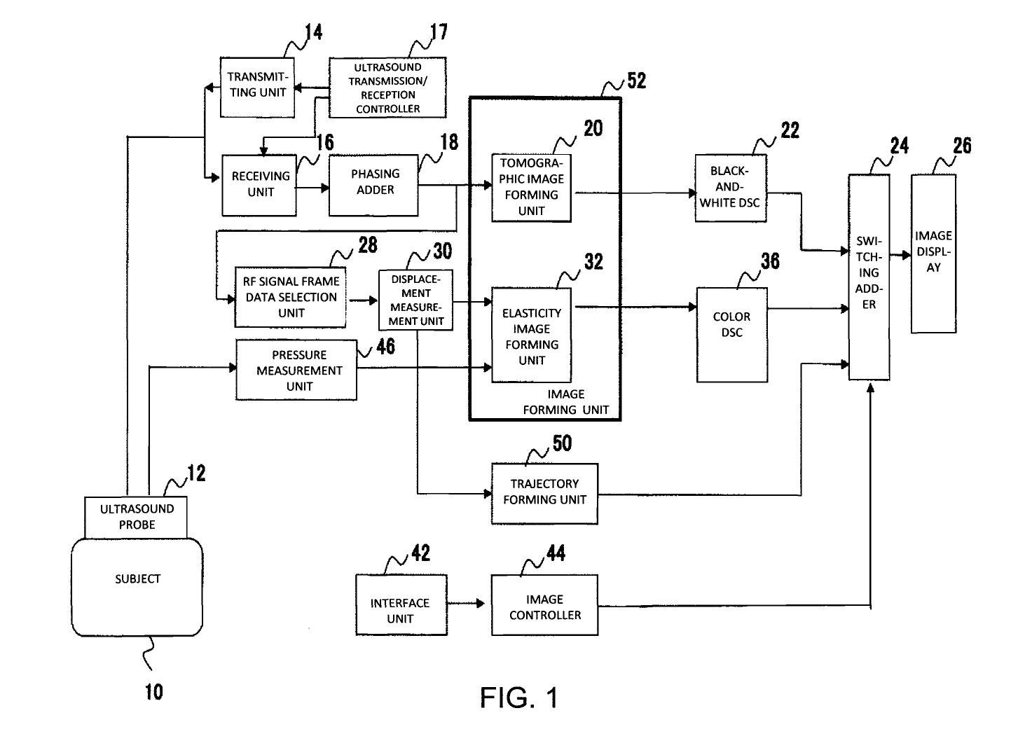

An ultrasound diagnostic apparatus according to the present invention will now be described with reference to the drawings. FIG. 1 is a block diagram exemplifying an ultrasound diagnostic apparatus according to a first preferred embodiment of the present invention.

As shown in FIG. 1, an ultrasound diagnostic apparatus according to the present embodiment comprises an ultrasound probe 12, a transmitting unit 14, a receiving unit 16, an ultrasound transmission/reception controller 17, a phasing adder 18, an RF signal frame data selection unit 28, a displacement measurement unit 30, a pressure measurement unit 46, an image forming unit 52, a black-and-white DSC (Digital Scan Converter) 22, a color DSC 36, a switching adder 24, an image display 26, and a trajectory forming unit 50. In addition, the image forming unit 52 forms an ultrasound image of a diagnosis site of a subject 10 through the ultrasound probe 12, and includes a tomographic image forming unit 20 and an elasticity image forming unit 32.

The ultrasound probe 12 is formed by placing a plurality of transducers, and transmits and receives ultrasound to and from the contacted subject 10 through the transducer. The transmitting unit 14 produces a transmission pulse for driving the ultrasound probe 12 to generate ultrasound, sets a point of conversion of the transmitted ultrasound at a certain depth, and repeatedly transmits the ultrasound with a certain time interval to the subject 10 through the ultrasound probe 12. The receiving unit 16 has functions to receive a generated time sequential reflection echo signal from the subject 10 through the ultrasound probe 12, and to amplify the received reflection echo signal with a predetermined gain to produce an RF signal (reception signal). The transmission/reception controller 17 controls the transmitting unit 14 and the receiving unit 16, to transmit and receive the ultrasound to and from the subject 10 through the ultrasound probe 12. The phasing adder 18 phase-adds the reflection echo signal received by the receiving unit 16. In this process, the phasing adder 18 receives an input of the RF signal amplified by the receiving unit 16 and phase-controls the RF signal, forms an ultrasound beam for one or a plurality of points of conversion, and time sequentially produces RF signal frame data which is ultrasound tomographic data.

The tomographic image forming unit 20 receives an input of the ultrasound tomographic data of the tomographic site of the subject 10; more specifically, the RF signal frame data from the phasing adder 18, applies signal processes such as gain correction, log compression, wave detection, outline emphasis, filter process, and the like, and forms a tomographic image (for example, black-and-white graded tomographic image of the subject 10).

The black-and-white DSC 22 comprises an A/D converter that converts the tomographic image data from the tomographic image forming unit 20 into a digital signal, a frame memory that time sequentially stores the plurality of converted tomographic image data, and a controlling controller. The black-and-white DSC 22 acquires the tomographic frame data in the subject 10 stored in the frame memory as one image, and reads the acquired tomographic frame data in television synchronization.

The RF signal frame data selection unit 28 stores the RF signal frame data which is output from the phasing adder 18, and selects at least two (a pair of) frame data from the group of stored group of RF signal frame data. For example, the RF signal frame data selection unit 28 sequentially stores the RF signal frame data produced in a time sequential manner; that is, based on the frame rate of the image, from the phasing adder 18, and selects the stored RF signal frame data (β) as first data and at the same time, selects one RF signal frame data (α) from among a group of RF signal frame data (β-1, β-2, β-3, . . . β-γ) stored in the past in the time sequence. The variables β, γ, and α are index numbers attached to the RF signal frame data, and are natural numbers.

The displacement measurement unit 30 measures a displacement of a living body tissue of the subject 10. More specifically, the displacement measurement unit 30 applies a one-dimensional or two-dimensional correlation process on the pair of data selected by the RF signal frame data selection unit 28; that is, the RF signal frame data (β) and the RF signal frame data (α), and determines a movement vector indicating a displacement in the living body tissue corresponding to each point of the tomographic image; that is, a one-dimensional or two-dimensional displacement distribution related to a direction and a magnitude of the displacement. Here, for the detection of the movement vector, a block matching method or a phase gradient method is employed.

In the block matching method, the image is divided into blocks made of, for example, N×N pixels (wherein N is a natural number), interest is focused on a block in a predetermined region (for example, on a parameter acquisition region to be described later), a block which is the most similar to the block of interest within the current frame is searched from previous frames, and a process for predictive coding referring to the found block; that is, a process for determining a sample value by a difference, is executed. With this process, the displacement of each point in the tomographic image is determined and the movement vector is detected. In the phase gradient method, an amount of movement of a wave is calculated based on phase information of the wave of the received signal to determine a displacement of each point in the tomographic image, and the movement vector is detected.

The pressure measurement unit 46 measures a stress at the measurement point in the subject 10 based on a pressure detected by a pressure sensor or the like provided between an ultrasound transmission/reception surface of the ultrasound probe 12 and the subject 10.

The elasticity image forming unit 32 determines a strain or a modulus of elasticity of the tissue at the tomographic site based on the ultrasound tomographic data of the tomographic site of the subject 10, and forms an elasticity image at the tomographic site based on the determined strain or modulus of elasticity.

In the present embodiment, the elasticity image forming unit 32 calculates the strain or modulus of elasticity of the living body tissue corresponding to each point in the tomographic image based on displacement information of the living body tissue measured by the displacement measurement unit 30; for example, the movement vector, using the RF signal frame data selected by the RF signal frame data selection unit 28, and forms an elasticity image signal; that is, elasticity frame data, based on the strain or the modulus of elasticity. In the calculation of the strain or the modulus of elasticity of the living body tissue, the elasticity image forming unit 32 also takes into consideration the pressure value which is output from the pressure measurement unit 46. In this case, the strain data is calculated by spatially differentiating the amount of movement of the living body tissue; for example, the displacement. The data of the modulus of elasticity is calculated by dividing a change of pressure by a change of the strain. For example, when the displacement measured by the displacement measurement unit 30 is L(α) and the pressure measured by the pressure measurement unit 46 is P(α), the strain ΔS(α) can be calculated by spatially differentiating L(α); that is, using following Equation (1):

ΔS(α)=ΔL(α)/Δα Equation (1)

The Young's modulus of the modulus Ym(α) of the modulus-of-elasticity data is determined by following Equation (2)

Ym(α)=ΔP(α)/ΔS(α) Equation (2)

Because the modulus of elasticity of the living body tissue corresponding to each point in the tomographic image is determined based on the Young's modulus Ym, two-dimensional elasticity image data can be consecutively obtained. Young's modulus refers to a ratio between a simple tensile stress applied on an object and strain generated in parallel with the direction of tension. The elasticity image forming unit 32 also includes a frame memory and an image processor, stores the elasticity frame data in the frame memory, and applies an image process on the stored frame data.

The color DSC 36 converts the output signal of the elasticity image forming unit 32 to a form matching the display on the image display 26. In other words, the color DSC 36 has a function to attach color phase information to the elasticity frame data which is output from the elasticity image forming unit 32, and converts the elasticity frame data into image data to which are added red (R), green (G), and blue (B) which are primary colors of the light. For example, the color DSC 36 converts elasticity data with a large strain into a red code, and converts elasticity data with a small strain into a blue code.

The switch adder 24 comprises a frame memory, an image processor, and an image selection unit, and produces a combined image or a parallel image of the tomographic image and the elasticity image through a method such as α-blending. The frame memory stores the tomographic image data from the black-and-white DSC 22 and the elasticity image data from the color DSC 36.

The image processor combines the tomographic image data and the elasticity image data stored in the frame memory while changing the combination ratio. The brightness information and the color phase information of each pixel of the combined image would be those obtained by adding the information of the black-and-white tomographic image and the color elasticity image with the combination ratio.

The image selection unit selects an image to be displayed from the tomographic image data and the elasticity image data in the frame memory and the combined image data of the image processor, and causes the image to be displayed on the image display 26. The switching adder 24 is controlled by a controller 44 based on an image display condition or the like which is set through an interface unit 42. The interface unit 42 includes an operation device such as a mouse, a keyboard, a trackball, a touch pen, a joystick, or the like, and is formed to allow input of the setting of the image display condition or the like through the operation device.

The image display 26 displays in a visible manner an image such as the tomographic image and the elasticity image or the like selected by the image selection unit of the switching adder 24, and a trajectory (two-dimensional displacement coordinates, a displacement histogram, or displacement-strain coordinates) formed by the trajectory forming unit 50 to be described later.

The trajectory forming unit 50 forms the trajectory related to the displacement of the region based on a displacement distribution in two-dimensional directions in an arbitrary region of the ultrasound image (tomographic image and elasticity image), and causes the trajectory to be displayed on the image display 26. A structure of the trajectory forming unit 50 which is a characteristic part of the present invention will now be described.

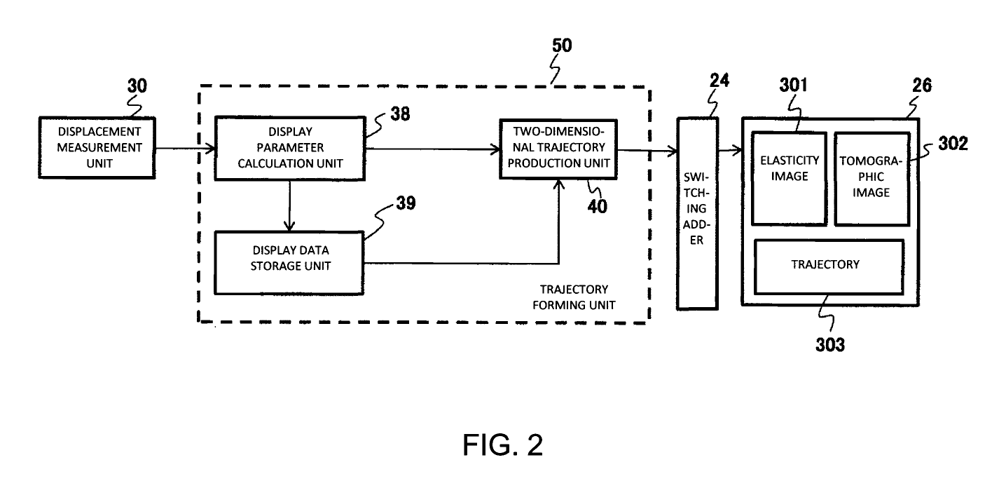

FIG. 2 is a block diagram exemplifying a structure of the trajectory forming unit 50 according to the present embodiment. As shown in FIG. 2, the trajectory forming unit 50 includes a display parameter calculation unit 38, a display data storage unit 39, and a two-dimensional trajectory production unit 40. In the present embodiment, the trajectory forming unit 50 time sequentially calculates, based on a displacement distribution in the two-dimensional directions in an arbitrary region of the ultrasound image, a parameter related to the displacement of the region, and forms a trajectory on a predetermined coordinate axis based on the calculated parameter.

The display parameter calculation unit 38 calculates a parameter related to a two-dimensional displacement distribution (displacement distribution in the X direction and Y direction) of a movement vector (vector showing a direction and a magnitude of the displacement in the living body tissue corresponding to each point in the tomographic image) determined in the displacement measurement unit 30.

The Y direction corresponds to a transmission direction of the ultrasound beam with respect to the living body tissue, and the X direction corresponds to a direction orthogonal to the Y direction on the tomographic image and the elasticity image displayed on the image display 26. In this case, the display parameter calculation unit 38 calculates a parameter (hereinafter referred to as a “displacement parameter”) related to the two-dimensional displacement distribution of the movement vector determined by the displacement measurement unit 30. The displacement parameter is calculated based on the two-dimensional distribution of the movement vector and as a statistical value, such as, for example, an average, a variance, a maximum, a minimum, a center value, a frequency, or the like, of displacement in two-dimensional directions (X direction and Y direction) in an arbitrary region (hereinafter referred to as a “parameter acquisition region”) in an image of at least one of the tomographic image and the elasticity image. The displacement represents a change of the displacement parameter of the parameter acquisition region from a point of time immediately before the current time to the current time.

The display data storage unit 39 time sequentially stores and holds the displacement parameter calculated by the display parameter calculation unit 38.

The two-dimensional trajectory production unit 40 forms a trajectory with respect to the two-dimensional directions based on the displacement parameter of the parameter acquisition region held in the display data storage unit 39, and causes the trajectory to be displayed on the image display 26 through the switching adder 24. Alternatively, the two-dimensional trajectory production unit 40 may form the trajectory based on the displacement parameter calculated by the display parameter calculation unit 38 in addition to or in place of the displacement parameter held in the display data storage unit 39. With this configuration, for example, the trajectory may be updated in real time based on the most recent displacement parameter. In the present embodiment, the two-dimensional trajectory production unit 40 forms the trajectory (two-dimensional displacement coordinates) by time sequentially plotting the displacement; that is, the displacement parameter, with respect to the two-dimensional directions of the parameter acquisition region, with the two-dimensional directions X direction and Y direction) as coordinate axes.

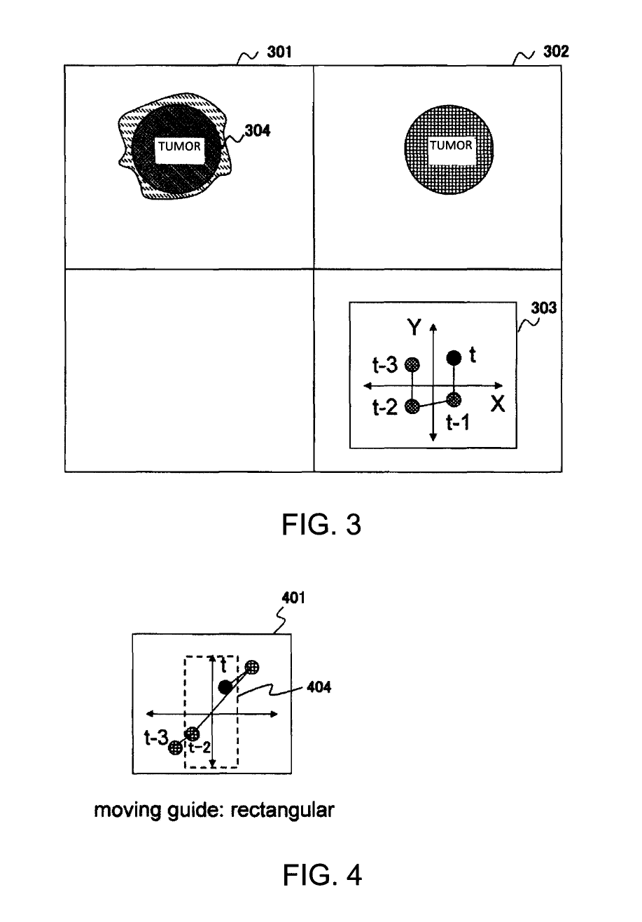

FIG. 3 is a diagram exemplifying displaying of an image on the image display 26 according to the present embodiment, and is a diagram showing a specific example display of an elasticity image 301, a tomographic image 302, and a trajectory 303 shown in FIG. 2. In this case, the trajectory forming unit 50 causes the trajectory (two-dimensional displacement coordinates) 303 of the displacement of the parameter acquisition region with respect to the two-dimensional directions to be displayed on the image display 26.

The trajectory 303 is displayed on the image display 26 along with the tomographic image 302 and the elasticity image 301. In other words, the trajectory forming unit 50 causes the trajectory 303 of the displacement with respect to the two-dimensional directions in the parameter acquisition region formed by the two-dimensional trajectory production unit 40 based on the displacement parameter of the parameter acquisition region to be displayed on the image display 26 along with the tomographic image 302 and the elasticity image 301. FIG. 3 shows an example in which the trajectory 303 is displayed with the tomographic image 302 and the elasticity image 301 in a tumor site.

The parameter acquisition unit for forming the trajectory 303 by the trajectory forming unit 50 is set for at least one image of the tomographic image 302 and the elasticity image 301. In this process, the setting of the parameter acquisition region can be achieved by, for example, a user designating a desired region in the tomographic image 302 or the elasticity image 301 displayed on the image display 26 using the operation device of the interface unit 42. The controller 44 can set a desired region on a tumor 304 which is a hard site to be particularly observed. For example, the controller 44 sets a region having a strain of less than or equal to a predetermined threshold, which forms a hard site, as the desired region.

Alternatively, the controller 44 sets a region having a modulus of elasticity of greater than or equal to a predetermined threshold, which forms a hard site, as the desired region. Thus, a desired region may be set not over the entirety of the image, but on the tumor 304 which is a hard site, and thus, a change with respect to time of the trajectory 303 of the hard site may be displayed on the image display 26. The operator can judge reliability of the elasticity image for the hard site to be particularly observed, based on the change with respect to time of the trajectory 303 of the hard site.

The trajectory 303 shown in FIG. 3 is formed by plotting displacement parameters of the past and present in the parameter acquisition region in the coordinate axes in the two-dimensional directions (XY coordinate axes). In this process, the number of plots of the displacement parameter is not particularly limited, and may be arbitrarily set, for example, according to the frame rate or the like for forming the tomographic image 302 or the elasticity image 301.

As an example, FIG. 3 shows the trajectory 303 formed by plotting the displacement parameter in the parameter acquisition unit for 4 points in time. In the trajectory 303, the current point of time is set as time t, and three points of time in the past from the time t are set, in order, as time t-1, time t-2, and time t-3. The time interval between each of these times may be set as identical to each other, or may alternatively be set different from each other.

In the trajectory 303, each of the plotted points of the times (displacement parameters) is connected by a straight line with an immediately preceding plotted point. Alternatively, the plotted points may be connected by, for example, an arrow line or the like directed from the previous plotted point to the next plotted point in place of the straight line, in order to allow the change with respect to time of the trajectory 303 to be understood at a glance.

In the trajectory 303, the plotted point of the current time t is displayed darker than the plotted points of the past times t-1˜t-3, and a display indicating which time the plotted point represents is also provided. The display form of the plotted points is not limited to such a configuration, and, for example, the plotted points of the current time t and the past times t-1˜t-3 may alternatively be displayed with different color phases, different sizes, etc.

Of the four coordinate regions separated by the X coordinate axis and the Y coordinate axis orthogonal to each other and shown in FIG. 3, a coordinate region in which the displacement parameter of the current time t is plotted is set as a first coordinate region, and, in a clockwise order from the first coordinate region, the coordinate regions are set as a second coordinate region, a third coordinate region, and a fourth coordinate region. In this case, the displacement parameters of the three times t-1, t-2, and t-3 are plotted in the second coordinate region, the third coordinate region, and the fourth coordinate region, respectively. Accordingly, it can be understood that the parameter acquisition region is displaced counterclockwise on the XY coordinate axes in the order of the fourth coordinate region, the third coordinate region, and the second coordinate region, and reaches the first coordinate region at the current time t. In other words, by observing the trajectory 303, it becomes possible to clearly understand in what direction on the XY coordinate axes the parameter acquisition region moves.

As shown in FIG. 3, the trajectory 303 is displayed along with the tomographic image 302 and the elasticity image 301, and the elasticity image 301 is formed basically based on the displacement in the Y direction. In other words, the elasticity image 301 is formed by executing a displacement calculation with respect to the Y direction corresponding to the transmission direction of the ultrasound beam to the living body tissue, and based on the calculation result of the strain or the modulus of elasticity determined from the displacement.

Therefore, if the trajectory 303 has a small displacement in the X direction and a large displacement in the Y direction, it can be judged that the strain, the modulus of elasticity, or the like of the parameter acquisition region forming the original data when the displacement parameter forming the trajectory 303 is calculated is highly reliable. In other words, for the trajectory 303 having a small displacement in the X direction and a large displacement in the Y direction, it can be judged that the elasticity image 301 displayed along with the trajectory 303 is formed with a high precision.

For example, when the strain of a tissue due to a body movement such as a heartbeat is to be diagnosed, the scanning direction of the ultrasound by the user may be adjusted so that the trajectory 303 is biased toward the Y direction and the data may be acquired, so that a higher precision elasticity image can be formed. Even in a case where the elasticity image is formed based on a lateral wave generated from inside and outside of the body of the subject, the reduction of the movement of the living body tissue in the lateral direction (displacement in the X direction) is important for obtaining stable elasticity information (strain, modulus of elasticity, etc.), and observation of such trajectory 303 contributes to this point. In addition, with the trajectory 303 having a small displacement in the X direction and a large displacement in the Y direction, it can be judged that the tomographic image 302 displayed along with the trajectory 303 is formed with high precision. This is because, in this case, it can be calculated that the error due to accumulation with time of the displacement in the X direction when the tomographic image 302 is formed is also small.

The ultrasound diagnostic apparatus of the present invention forms the trajectory related to the displacement of an arbitrary region of the ultrasound image based on the displacement distribution in the two-dimensional directions. The ultrasound diagnostic apparatus includes the image forming unit 52 (tomographic image forming unit 20 and elasticity image forming unit 32) that forms an ultrasound image of the diagnosis site of the subject through the ultrasound probe 12, the image display 26 that displays the ultrasound image, and the trajectory forming unit 50 (display parameter calculation unit 38, display data storage unit 39, and two-dimensional trajectory production unit 40) that forms a trajectory related to the displacement of the region based on the displacement distribution in the two-dimensional directions in the arbitrary region of the ultrasound image, and that causes the trajectory to be displayed on the image display 26.

A trajectory display method according to the present invention includes a step of forming an ultrasound image of a diagnosis site of the subject 10 through the ultrasound probe 12; a step of forming, based on a displacement distribution in the two-dimensional directions in an arbitrary region of the ultrasound image, a trajectory related to a displacement of the region; and a step of displaying the ultrasound image and the trajectory.

### Second Preferred Embodiment

An ultrasound diagnostic apparatus according to a second preferred embodiment of the present invention will now be described with reference to the drawings. Unless otherwise particularly stated, structures are similar to those of the ultrasound diagnostic apparatus of the first preferred embodiment.

In the present embodiment, a trajectory (two-dimensional displacement coordinates) including a predetermined guide is displayed to notify an appropriate displacement range of the parameter acquisition region to the user. FIGS. 4-6 are diagrams exemplifying trajectories (two-dimensional displacement coordinates) 401-403 in the present embodiment. In the present embodiment, the trajectory forming unit 50 (FIG. 1) forms the trajectories 401-403 of the displacement (displacement parameter) in the two-dimensional directions of the parameter acquisition region, and causes the trajectories to be displayed on the image display 26. The trajectories 401-403 include guides 404-406 indicating appropriate displacement ranges of the parameter acquisition region. The guides 404-406 are visible information including at least one of a text, a diagram, and a sign indicating an appropriate displacement range in the two-dimensional directions in the parameter acquisition region.

With such a configuration, when the plotted points of the trajectories 401-403 fall within the ranges indicated by the respective guides 404-406, the user can understand that the displacement of the parameter acquisition region is appropriately captured; that is, data acquisition is appropriately performed. As a result, the user can confirm that the tomographic image and the elasticity image (for example, the tomographic image 302 and the elasticity image 301 shown in FIG. 3) displayed along with the trajectories 401-403 are formed with a high precision.

On the other hand, if the plotted points of the trajectories 401-403 are out of the ranges indicated by the guides 404-406, the user can understand that the displacement of the parameter acquisition region is not necessarily appropriately captured; that is, there is a possibility that the data acquisition is not appropriately performed. As a result, the user can judge that the image precision of the tomographic image and the elasticity image displayed along with the trajectories 401-403 may be low. In this case, the user can again acquire the data, or the like, so that the plotted points of the trajectories 401-403 fall within the ranges indicated by the guides 404-406. In other words, the guides 404-406 contribute to improving the image precision of the tomographic image and the elasticity image.

As shown in FIG. 4, the trajectory 401 includes the guide 404. In this case, the guide 404 is a rectangle longer in the Y direction than the x direction, and indicates that, in the Y direction, a relatively large displacement is appropriate, while in the x direction, only a relatively small displacement is appropriate. The guide 404 may include text information showing the shape (for example, “moving guide: rectangular”).

Therefore, the guide 404 is information suitable, for example, for understanding the image precision of the elasticity image 301 (FIG. 3) and for improving the image precision. In the trajectory 401 shown in FIG. 4, each of the plotted points of the four points in time (t, t-1˜t-3) falls within the appropriate displacement range in the Y direction indicated by the guide 404, but the plotted points of time t-1 and the time t-3 do not fall within the appropriate displacement range in the X direction indicated by the guide 404. According to such a configuration, the user can understand that the parameter acquisition region is displaced in the X direction exceeding the appropriate range in the time t-1 and the time t-3.

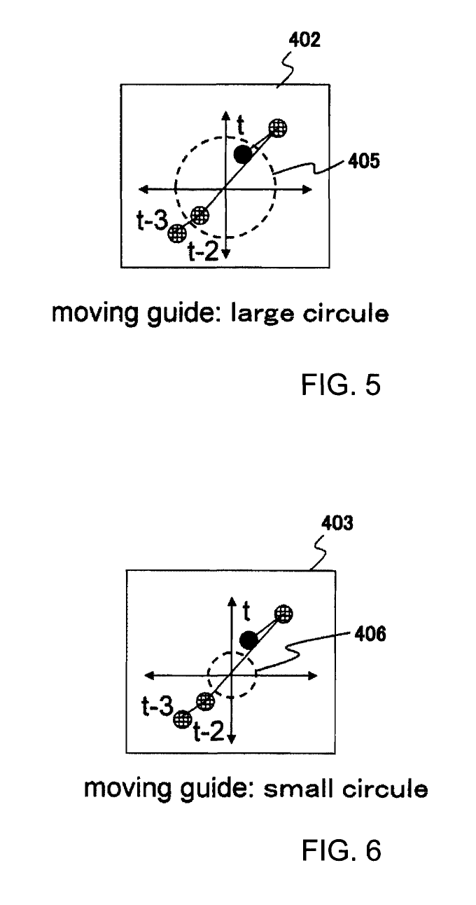

Similarly, as shown in FIG. 5, the trajectory 402 includes the guide 405. In this case, the guide 405 is a circle centered at an intersection of the XY coordinate axes (origin), and indicates that a displacement falling within the circle is appropriate. The guide 405 may include text information showing the shape (for example, “moving guide: large circle”).

Therefore, the guide 405 is information suitable, for example, for understanding an image precision of the tomographic image 302 (FIG. 3); in particular, a graded image using a contrast medium, and for improving the image precision. In the trajectory 402 shown in FIG. 5, of the plotted points of the four points in time (t, t-1˜t-3), the plotted points of the current time t and the time t-2 fall within the appropriate displacement range circle indicated by the guide 405, but the plotted points of the time t-1 and the time t-3 are out of the circle indicated by the guide 405 and do not fall within the appropriate displacement range. Accordingly, the user can understand that the parameter acquisition region is displaced outside the appropriate range at the time t-1 and the time t-3.

Similarly, as shown in FIG. 6, the trajectory 403 includes the guide 406. In this case, the guide 406 has a circular shape having a smaller radius than the guide 405 and centered at the intersection of the XY coordinate axes (origin). Because of this, the guide 406 indicates that a displacement falling within a smaller circle than the guide 405 is appropriate, and the guide 406 is a guide having a narrower appropriate range than the guide 405. The guide 406 may include text information showing the shape (for example, “moving guide: small circle”).

Therefore, the guide 406 is suitable as a guide, for example, for more strictly understanding the image precision of the tomographic image 302 (FIG. 3); in particular, the graded image using the contrast medium, and for improving the image precision. In the trajectory 403 shown in FIG. 6, the plotted points of the four points in time (t, t-1˜t-3) do not fall within the appropriate displacement range in the circle indicated by the guide 406. Accordingly, the user can understand that the parameter acquisition region is displaced exceeding the appropriate range at all of the four points in time (t, t-1˜t-3).

Here, the guides 404-406 may be displayed with the trajectories 401-403, for example, according to the mode of the image (elasticity image, tomographic image, or the like) to be displayed on the image display 26, and the living body tissue to be diagnosed (tumor site, liver site, mammary gland site, prostate site, or the like). In this process, the guides 404-406 may be held in the display data storage unit 39 of the trajectory forming unit 50 in advance, and may be fittingly formed in a manner to be included in the trajectories 401-403 by the two-dimensional trajectory production unit 40.

Alternatively, the trajectory forming unit 50 can form the trajectories 401-403 with different display forms between the plotted points that are within the appropriate displacement ranges indicated by the guides 404-406 and the plotted points outside of the ranges. For example, the trajectory forming unit 50 may display the plotted points falling within the ranges indicated by the guides 404-406 in an emphasized manner such as in a darker color or a red color, or display the plotted points that do not fall within the ranges indicated by the guides 404-406 in an emphasized manner such as in a darker color and a red color.

Alternatively, the trajectory forming unit 50 may remove the trajectory including plotted points (display parameters) that do not fall within the appropriate displacement range indicated by the guides 404-406, select the trajectory formed by only the plotted points (displacement parameters) falling within the appropriate displacement ranges indicated by the guides 404-406, and output the selected trajectories to the switching adder 24 (FIG. 1). With such a configuration, the image data may be held in a cine memory while removing image data of the elasticity image, the tomographic image, or the like synchronized with the removed trajectory. As a result, it becomes possible to display only the trajectory formed only by the plotted points (displacement parameters) falling within the ranges indicated by the guides 404-406, and the image data of the elasticity image, the tomographic image, or the like synchronized with the trajectory on the image display 26 automatically or manually by the user, at a timing of freeze or the like. With such a configuration, the diagnosis efficiency of the ultrasound diagnostic apparatus can be improved.

### Third Preferred Embodiment

An ultrasound diagnostic apparatus according to a third preferred embodiment of the present invention will now be described with reference to the drawings. Unless otherwise particularly stated, structures are similar to those of the ultrasound diagnostic apparatus of the first preferred embodiment.

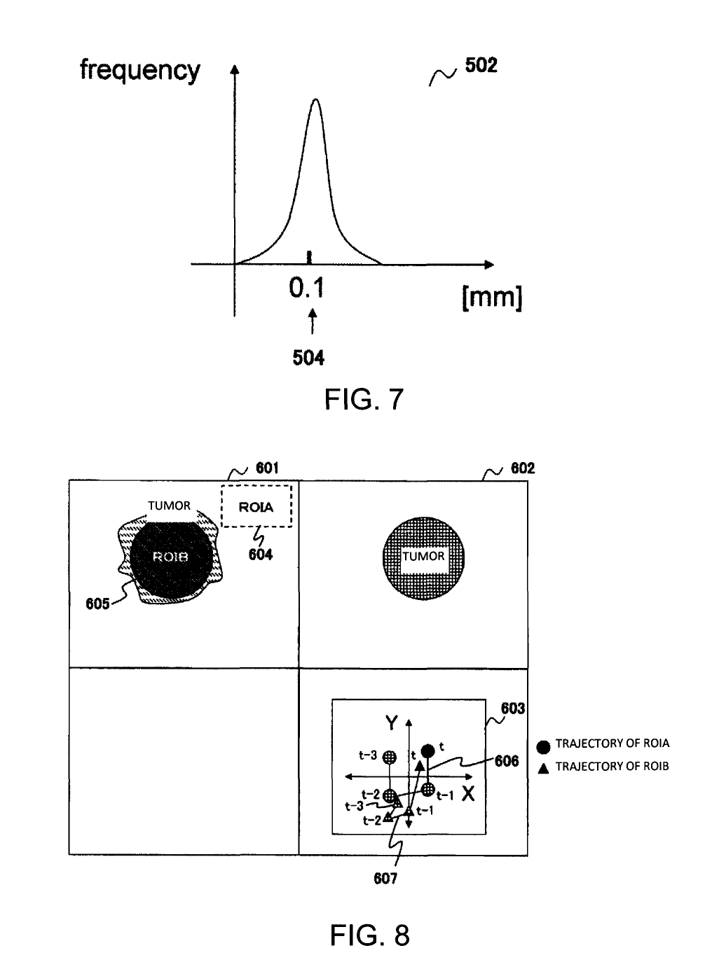

In the present embodiment, in addition to the trajectory (two-dimensional displacement coordinates) including the predetermined guide of the second preferred embodiment described above, a graph showing a relationship between a magnitude and a frequency of the displacement (hereinafter referred to as a “displacement histogram”) is displayed on the image display 26 (FIG. 1) as the trajectory. FIG. 7 is a diagram exemplifying a displacement histogram 502 which is a trajectory in the present embodiment. In the present embodiment, the trajectory forming unit 50 (FIG. 1) forms the trajectory (as an example, a two-dimensional displacement coordinate 402 shown in FIG. 5) of the displacement (displacement parameter) in two-dimensional directions in the parameter acquisition region, and causes the trajectory to be displayed on the image display 26, and at the same time, causes a guide (as an example, the guide 405 shown in FIG. 5) indicating an appropriate displacement range of the parameter acquisition region to be displayed on the image display 26. In this case, the guide 405 has a circular shape centered at the intersection of the XY coordinate axes (origin), and indicates a displacement falling within the circle to be appropriate.

In the present embodiment, as shown in FIG. 7, the trajectory forming unit 50 forms the trajectory (displacement histogram) 502 showing the relationship between the magnitude and frequency of the displacement (displacement parameter) in the two-dimensional directions in the parameter acquisition region, and causes the trajectory 502 to be displayed on the image display 26.

More specifically, the display parameter calculation unit calculates a parameter (hereinafter referred to as a “displacement frequency parameter”) showing the relationship between the magnitude and frequency of the displacement in the two-dimensional directions in the parameter acquisition region, based on a two-dimensional distribution of the movement vector determined by the displacement measurement unit 30 (FIG. 1). The display data storage unit 39 time sequentially stores and holds the displacement frequency parameter. The two-dimensional trajectory production unit 40 forms the trajectory (displacement histogram) 502 showing the relationship between the displacement in the two-dimensional directions and the frequency in the parameter acquisition region based on the displacement frequency parameters in the present and in the past, with the coordinate axes being an axis showing the magnitude of the displacement (displacement axis) and an axis showing the frequency at which the displacement is measured (frequency axis), and causes the trajectory 502 to be displayed on the image display 26 through the switching adder 24.

The trajectory 502 includes a displacement axis (horizontal axis) showing the displacement of the displacement parameter from the origin, and a frequency axis (vertical axis) showing the frequency of the display parameter for the displacement. In addition, the trajectory 502 includes a guide 504 indicating an appropriate displacement range of the parameter acquisition region.

The guide 504 is visible information including at least one of a text, a figure, and a sign indicating the appropriate displacement range in the two-dimensional directions in the parameter acquisition region. In this case, on the displacement axis, an appropriate displacement point of the parameter acquisition region based on the guide 405 (FIG. 5) of the trajectory 402 is shown as the guide 504. The displacement point that becomes the guide 504 may be arbitrarily set and shown based on the guide 405.

As an example, in the trajectory 502 shown in FIG. 7, the displacement point is shown as 0.1 mm. In other words, the guide 405 shown in FIG. 5 indicates that a displacement within a circle having a radius of 0.1 mm and centered at the intersection of the XY coordinate axes (origin) is appropriate. By observing the trajectory 502, it can be understood that almost a half of the displacement frequency parameter falls within the appropriate displacement range indicated by the guide 504. On the other hand, it can also be understood that the remaining half of the displacement frequency parameter does not fall within the appropriate displacement range indicated by the guide 504, and is displaced exceeding the appropriate displacement range.

Specifically, it can be easily judged how often the displacement parameter falls within the displacement allowance range indicated by the guide 504. The number of plotted points (displacement parameters) of the trajectory 402 and the number of samples of the displacement frequency parameter of the trajectory 502 may be the same or may differ from each other. For example, the trajectory 402 can form as the plotted points the displacement parameters of four immediately near points in time in the displacement frequency parameter of the trajectory 502. In this case, the trajectory 502 can show the relationship between the displacement and the frequency of the displacement parameter from the data acquisition to the current point in time.

### Fourth Preferred Embodiment

An ultrasound diagnostic apparatus according to a fourth preferred embodiment of the present invention will now be described with reference to the drawings. Unless otherwise particularly stated, structures are similar to those of the ultrasound diagnostic apparatus of the first preferred embodiment.

In the present embodiment, trajectories (two-dimensional displacement coordinates) of a plurality of parameter acquisition regions are displayed on the image display 26 (FIG. 1) along with the elasticity image and the tomographic image. FIG. 8 is a diagram exemplifying displaying of an image on the image display 26 in the present embodiment. In this case, the trajectory forming unit 50 (FIG. 1) causes a trajectory (two-dimensional displacement coordinates) 603 of the displacement in the two-dimensional directions in a plurality of parameter acquisition regions to be displayed on the image display 26. The trajectory 603 is displayed on the image display 26 along with a tomographic image 602 and an elasticity image 601.

FIG. 8 shows an example in which the trajectory 603 in two parameter acquisition regions is displayed along with the tomographic image 602 and the elasticity image 601 in a tumor site. The trajectory 603 includes a trajectory 606 in an ROIA 604, which is a parameter acquisition region, and a trajectory 607 in an ROIB 605, which is a different parameter acquisition region. The trajectory 606 of the ROIA 604 is formed by plotting the displacement parameters in the ROIA 604 in the present and in the past on the coordinate axes (XY coordinate axes) in the two-dimensional directions. The trajectory 607 of the ROIB 605 is formed by plotting the displacement parameters in the ROIB 605 in the present and in the past in the coordinate axes in the two-dimensional directions (XY coordinate axes) identical to those of ROIA 604.

In FIG. 8, the plotted point of the trajectory 606 in the ROIA 604 is shown by a circular mark and the plotted point of the trajectory 607 in the ROIB 605 is shown by a triangular mark. The trajectory 606 of the ROIA 604 and the trajectory 607 of the ROIB 605 may alternatively be formed by plotting the displacement parameters not on the same coordinate axes but on individual coordinate axes, and displayed.

The ROIA 604 and the ROIB 605 which are parameter acquisition regions are set for the elasticity image 601. In this case, the ROIA 604 is set for a near site of the tumor site (for example, a fat site), and the ROIB 605 is set for the tumor site. The setting of the ROIA 604 and the ROIB 605 may be achieved, for example, by the user designating a desired region on the elasticity image 601 displayed on the image display 26 using the operation device of the interface unit 42. In addition, in the present embodiment, the ROIA 604 and the ROIB 605 are set for the elasticity image 601, but alternatively, the ROIA 604 and the ROIB 605 may be set for the tomographic image 602 or for both the elasticity image 601 and the tomographic image 602.

By displaying the trajectory 603 of a plurality of parameter acquisition regions as in the present embodiment, it becomes possible to more reliably judge that the elasticity image 601 and the tomographic image 602 displayed along with the trajectory 603 are formed with a high precision. For example, depending on the structure of the living body tissue, the displacement direction in the living body tissue may become uneven, and, in this case, the two-dimensional displacement distribution of the movement vector in the living body tissue becomes unstable. Thus, in such a case, the image precision of the elasticity image of the living body tissue is reduced, and the trajectory formed using the inside of the living body tissue as the parameter acquisition region is not appropriate. In order to avoid such a circumstance, in the present embodiment, the trajectory 603 of a plurality of parameter acquisition regions is set to be observable.

Specifically, when both the trajectory 606 of the ROIA 604 and the trajectory 607 of the ROIB 605 are trajectories having a small displacement in the X direction and a large displacement in the Y direction, the displacement directions of the ROIA 604 and the ROIB 605 which are set distanced from each other are uniform, and it can be judged that the trajectory 603 is appropriately formed. As a result, it can be judged that the elasticity image 601 and the tomographic image 602 displayed along with the trajectory 603 are formed with high precision. On the contrary, when at least one of the trajectory 606 of the ROIA 604 and the trajectory 607 of the ROIB 605 is not a trajectory having a small displacement in the x direction and a large displacement in the Y direction, it can be judged that the displacement directions of the ROIA 604 and the ROIB 605 which are set distanced from each other are not uniform. In this case, the user can again acquire the data so that both trajectories are biased toward the displacement in the Y direction. With such a configuration, for example, when strain ratio of a plurality of living body tissues or the like is to be measured, the strain ratio can be calculated based on the strains with high reliability and in which the two-dimensional displacement distribution of the movement vector in the living body tissue is stable.

Alternatively, in the present embodiment, there may be employed a configuration in which a trajectory (two-dimensional displacement coordinates) including guides similar to the guides 404-406 of the above-described second preferred embodiment is displayed, and the appropriate displacement ranges of the parameter acquisition regions (ROIA 604 and ROIB 605) may be notified to the user. With such a configuration, it becomes possible to more reliably judge whether or not the elasticity image 601 and the tomographic image 602 displayed along with the trajectory 603 are formed with high precision. Alternatively, in the present embodiment, a trajectory (displacement histogram) showing the relationship between the magnitude and frequency of displacement may be formed for the trajectory 606 and the trajectory 607 similar to the above-described third preferred embodiment, and displayed along with the trajectory 606 and the trajectory 607.

### Fifth Preferred Embodiment

An ultrasound diagnostic apparatus according to a fifth preferred embodiment of the present invention will now be described with reference to the drawings. Unless otherwise particularly stated, structures are similar to those of the ultrasound diagnostic apparatus of the first preferred embodiment.

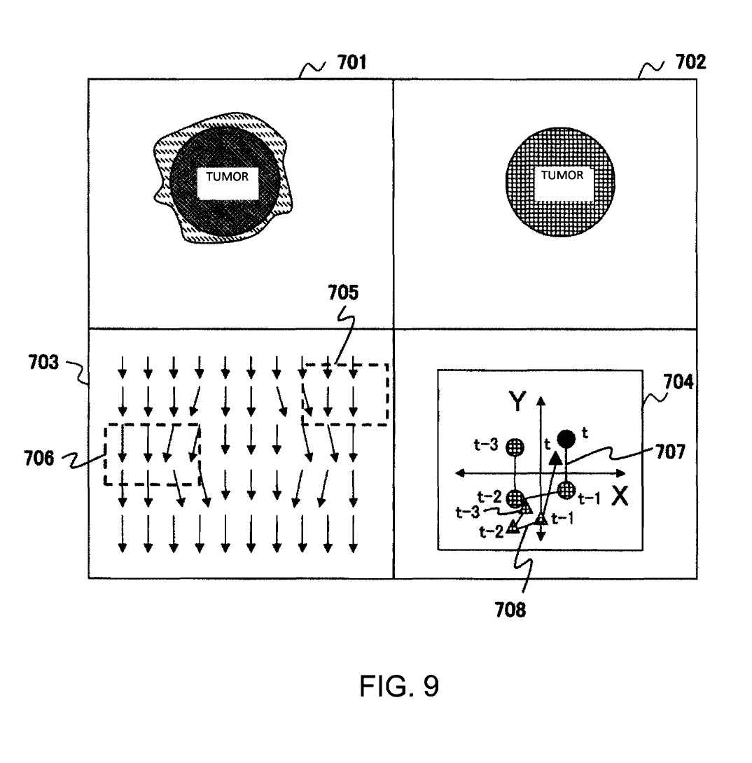

In the present embodiment, a trajectory (two-dimensional displacement coordinates) of the parameter acquisition region is displayed on the image display 26 (FIG. 1) along with a two-dimensional displacement image in addition to the elasticity image and the tomographic image. FIG. 9 is a diagram exemplifying displaying of an image on the image display 26 in the present embodiment. In this case, the trajectory forming unit 50 (FIG. 1) causes a trajectory (two-dimensional displacement coordinates) 704 of a displacement of the parameter acquisition region with respect to the two-dimensional directions to be displayed on the image display 26. The trajectory 704 is displayed on the image display 26 along with a two-dimensional displacement image 703 in addition to a tomographic image 702 and an elasticity image 701. FIG. 9 shows an example in which the trajectory 704 in two parameter acquisition regions is displayed along with the tomographic image 702, the elasticity image 701, and the two-dimensional displacement image 703 in a tumor site. In other words, the present embodiment shows an example image display in which the two-dimensional displacement image 703 is added to the example image display of the above-described fourth preferred embodiment (FIG. 8).

In this case, the trajectory 704 includes a trajectory 707 and a trajectory 708 respectively in an ROIA 705 and an ROIB 706 which are different parameter acquisition regions. The ROIA 705 and the ROIB 706 are set for the two-dimensional displacement image 703. In this regard, the present embodiment differs from the fourth preferred embodiment in which the parameter acquisition regions (ROIA 604 and ROIB 605) are set for the elasticity image 601. The setting of the ROIA 705 and the ROIB 706 can be achieved by, for example, the user designating a desired region in the two-dimensional displacement image 703 displayed on the image display 26 using the operation device of the interface unit 42.

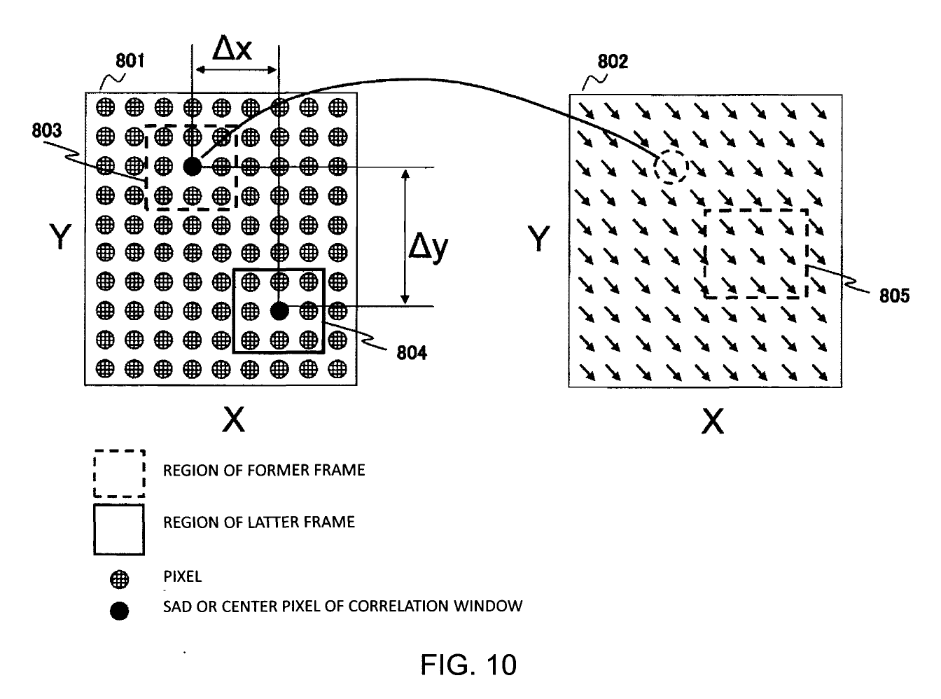

The two-dimensional displacement image will now be described. FIG. 10 is a schematic diagram showing a displacement detection method in the displacement measurement unit 30 (FIG. 1) when the two-dimensional displacement image is to be formed. The displacement measurement unit 30 detects for each point (pixel) of the tomographic image a displacement in the Y direction necessary for forming the elasticity image of the living body tissue and a displacement in the x direction for tracking a lateral movement of the received signal. As shown in FIG. 10, the displacement measurement unit 30 can detect the displacements in the X direction and in the Y direction by applying, in predetermined RF signal frame data (former frame) and RF signal frame data which is past in time in relation to the RF signal frame data (latter frame), a calculation such as SAD (Sum of Absolute Difference) and self-correlation on a movement region in the latter frame with respect to an arbitrary region of the former frame.

For example, a case is considered in which, in a region 801 (pixel region of 9×10) including 9 pixels in the X direction and 10 pixels in the Y direction shown in FIG. 10, a region 803 in the former frame surrounded by a broken line has moved to a region 804 in the latter frame surrounded by a solid line. In this case, a center point (point shown by a dark color in the broken line) of the region 803 in the former frame has moved by Δx in the X direction and Δy in the Y direction in the latter frame, and becomes the center point (point shown by a dark color in the solid line) of the region 804. An image is formed that shows for each pixel a displacement from the former frame to the latter frame of the pixel of the pixel region 801; that is, the direction and magnitude of the current displacement, as a movement vector.

In this manner, the two-dimensional displacement image 802 is formed. As an example, in the two-dimensional displacement image 802, the displacements from the former frame to the latter frame of the pixels of the pixel region 801 are in a displacement state shown by the movement vectors of approximately the same magnitude and toward the down and right direction for each pixel. With the two-dimensional displacement image 802, for example, the displacement state of the region 805 may be understood as the state (direction, magnitude, variation, etc.) of the movement vector.

The two-dimensional displacement image 802 is formed as one elasticity image by the elasticity image forming unit 32 (FIG. 1) based on the movement vector measured by the displacement measurement unit 30. The formed two-dimensional displacement image 802 is displayed on the image display 26 by the elasticity image forming unit 32 through the color DSC unit 36 and the switching adder 24.

In the present embodiment, the displacement measurement unit 30 (FIG. 1) detects the displacement in the X direction and the displacement in the Y direction at each point (pixel) of the tomographic image 702, and measures the movement vector. The elasticity image forming unit 32 (FIG. 1) forms the two-dimensional displacement image 703 based on the movement vector measured by the displacement measurement unit 30, and causes the two-dimensional displacement image 703 to be displayed on the image display 26 through the color DSC unit 36 and the switching adder 24. With such a configuration, the trajectory 704 (the trajectory 707 in the ROIA 705 and the trajectory 708 in the ROIB 706) can be displayed on the image display 26 (FIG. 1) along with the elasticity image 701, the tomographic image 702, and additionally, the two-dimensional displacement image 703.

As described above, in the present embodiment, the two-dimensional displacement image 703 is displayed, and the ROIA 705 and the ROIB 706 are set for the two-dimensional displacement image 703. Because of this, the ROIA 705 and the ROIB 706, which are parameter acquisition regions, can be set while checking the displacement distribution shown on the two-dimensional displacement image 703. Therefore, the precision of the trajectory 704 showing the displacement (displacement parameter) for the two-dimensional directions of the ROIA 705 and the ROIB 706 can be improved. In other words, displacements of the ROIA 705 and the ROIB 706 can be accurately captured.

### Sixth Preferred Embodiment

An ultrasound diagnostic apparatus according to a sixth preferred embodiment of the present invention will now be described with reference to the drawings. Unless otherwise particularly stated, structures are similar to those of the ultrasound diagnostic apparatus according to the first preferred embodiment.

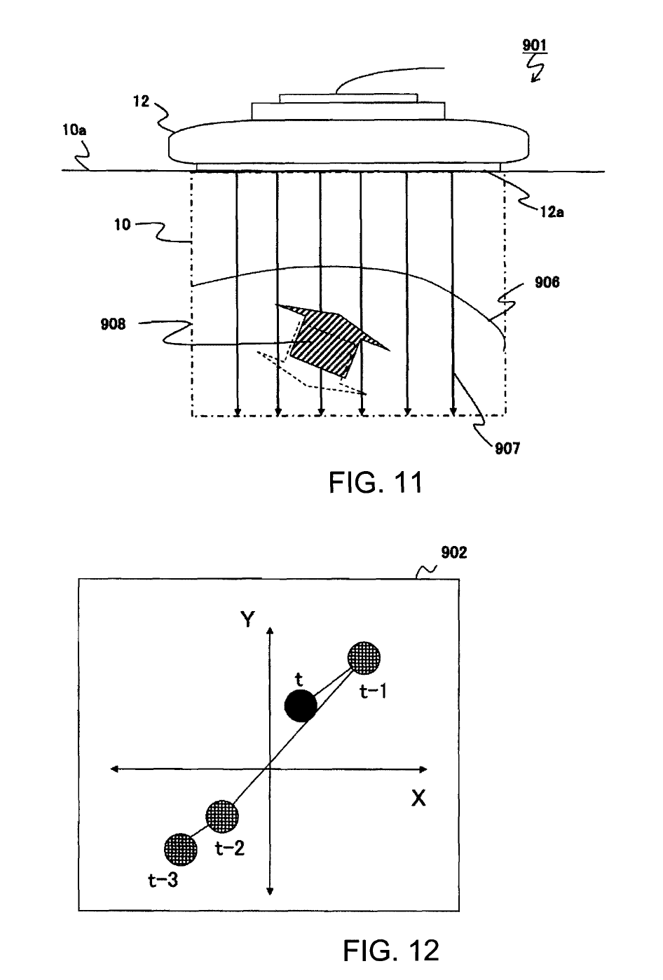

In the present embodiment, a displacement direction of a parameter acquisition region is calculated from a trajectory (two-dimensional displacement coordinates) of the parameter acquisition region, and a transmission direction of ultrasound transmitted from the ultrasound probe 12 (FIG. 1) (hereinafter referred to as an “ultrasound scanning direction”) is changed based on the calculated displacement direction. FIGS. 11-15 are schematic diagrams for explaining the calculation of the displacement direction and the change of the ultrasound scanning direction in the present embodiment.

As an example, there is considered a case in which the parameter acquisition region is set on an organ such as a liver on the ultrasound image, the trajectory of the organ is formed, and the trajectory is displayed and observed on the image display 26 (FIG. 1) along with the ultrasound image (elasticity image and tomographic image). Ina state 901 shown in FIG. 11, a displacement of the organ is detected.

In this case, an ultrasound scanning direction 907 of the ultrasound probe 12 is set in a vertical direction with respect to a probe surface 12a (or, from another perspective, a body surface 10a of the subject 10). With such a configuration, the ultrasound probe 12 transmits the ultrasound through the plurality of transducers in the ultrasound scanning direction 907 to an organ 906 of the subject 10 to be observed.

Meanwhile, the organ 906 to be observed is displaced (contraction and dilation) by the heartbeat in a direction 908 inclined by a predetermined angle (for example, angle θ shown in FIG. 14) with respect to the ultrasound scanning direction 907. As described, when the displacement of the organ 906 is detected using the heartbeat, the displacement direction 908 does not necessarily coincide with the ultrasound scanning direction 907. This is because the direction is affected by the contact state of the ultrasound probe 12 on the body surface 10a and the structure of the organ 906. In consideration of this, in the present embodiment, the ultrasound scanning direction is made to coincide with the displacement direction 908 of the organ 906.

In the present embodiment, the trajectory forming unit 50 (FIG. 1) forms a trajectory (two-dimensional displacement coordinates) 902 of the displacement parameter in the parameter acquisition region which is set for the organ 906, and causes the trajectory 902 to be displayed on the image display 26 (FIG. 12). In this process, with the trajectory 902, the trajectory forming unit 50 calculates, for example, an angle of the plotted point of the trajectory 902 with respect to the Y coordinate axis in an arbitrary set time period (as an example, elapsed time from time t-3 to current time t), and calculates an average of the calculated angles for the plotted points. The trajectory forming unit 50 calculates the calculated average of the angle as an inclination angle of the organ 906 with respect to the ultrasound scanning direction 907 (hereinafter referred to as a “displacement direction angle”).

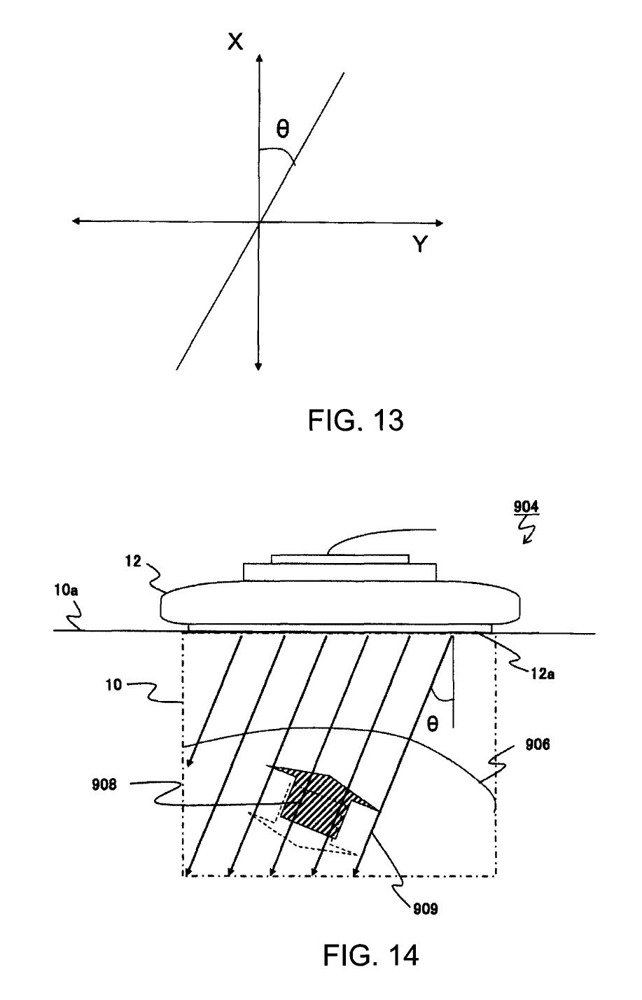

For example, the displacement direction angle in the trajectory 902 can be calculated as θ in a two-dimensional displacement coordinate 903 shown in FIG. 13. By calculating the displacement direction angle θ, it becomes possible to calculate the displacement direction of the organ 906 as a direction inclined from the ultrasound scanning direction 907 by the displacement direction angle θ.

With such a configuration, the transmission angle of the ultrasound (ultrasound scanning direction 907) transmitted from the ultrasound probe 12 can be automatically changed based on the displacement direction angle θ calculated by the trajectory forming unit 50. More specifically, a delay control can be applied on the transmitting unit 14 by the ultrasound transmission/reception controller 17 (FIG. 1), to transmit the ultrasound from the transmitting unit 14 through the ultrasound probe 12 in a direction inclined by the displacement direction angle θ from the ultrasound scanning direction 907, as shown by a state 904 in FIG. 14. In this case, the ultrasound probe 12 transmits the ultrasound through the plurality of transducers in an ultrasound scanning direction 909 to the organ 906 of the subject 10 to be observed. Therefore, the ultrasound scanning direction 909 and the displacement direction 908 of the organ 906 by the heartbeat can be made to coincide.

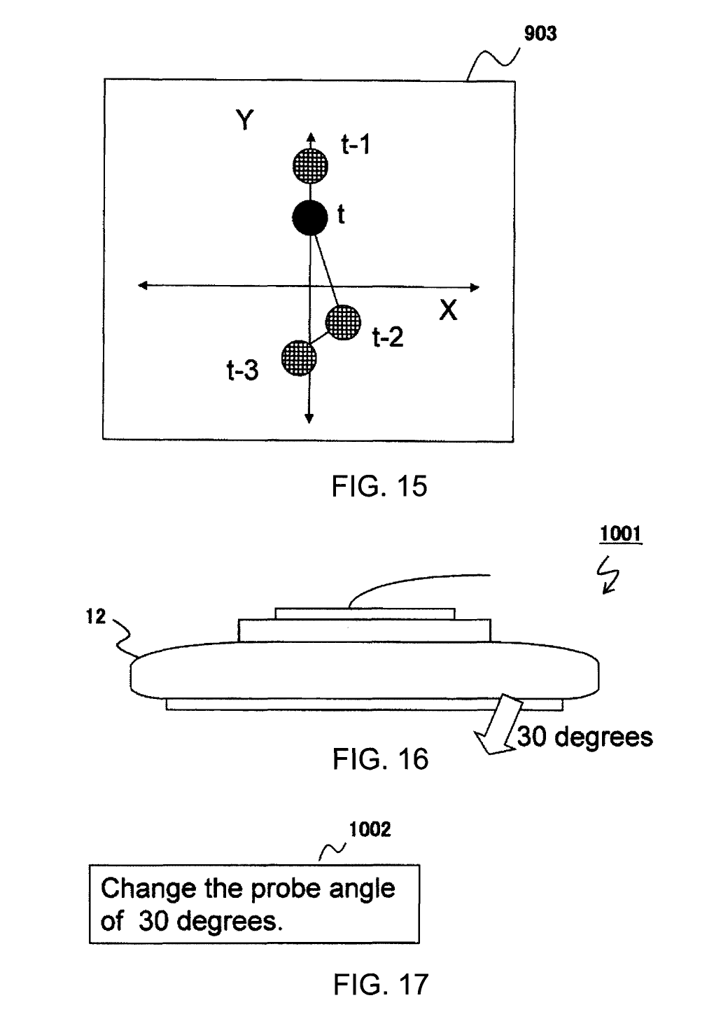

In a state where the ultrasound scanning direction 909 and the displacement direction 908 are made to coincide in this manner, the trajectory forming unit 50 forms a trajectory (two-dimensional displacement coordinates) 905 of the displacement parameter in the parameter acquisition region which is set for the organ 906, and causes the trajectory 905 to be displayed on the image display 26 (FIG. 15). In this case, the trajectory 905 is a trajectory having a small displacement in the X direction and a large displacement in the Y direction. In other words, the trajectory 905 is biased toward the Y direction, and the elasticity image and the tomographic image having a high image precision can be displayed along with the trajectory 905. From another perspective, because the transmission angle of the ultrasound transmitted from the ultrasound probe 12 is automatically changed so that the trajectory 905 is biased along the Y direction, the user can more intuitively judge the image precision of the elasticity image and the tomographic image.

### Seventh Preferred Embodiment

An ultrasound diagnostic apparatus according to a seventh preferred embodiment of the present invention will now be described with reference to the drawings. Unless otherwise particularly stated, structures are similar to those of the ultrasound diagnostic apparatus according to the first preferred embodiment.

In the present embodiment, a displacement direction of a parameter acquisition region is calculated from a trajectory (two-dimensional displacement coordinates) of the parameter acquisition region, and a guide and a message related to the displacement direction are displayed on the image display 26 (FIG. 1). The guide and message are visible information including at least one of a text, a figure, and a sign related to the displacement direction of the parameter acquisition region. The present embodiment is an alternative configuration of the above-described sixth preferred embodiment, and the displacement direction (from another perspective, a displacement direction angle θ shown in a two-dimensional displacement coordinate 903 of FIG. 13) is calculated in a manner similar to that in the sixth preferred embodiment. FIG. 16 is a diagram exemplifying a guide in the present embodiment, and FIG. 17 is a diagram exemplifying a message in the present embodiment. In this case, the trajectory forming unit 50 forms a guide 1001 and a message 1002, or the like based on the calculated displacement direction angle θ, and causes the guide and message to be displayed on the image display 26 through the switching adder 24.

For example, the guide 1001 is formed by combining a mark showing the ultrasound probe 12 (FIG. 1), an arrow showing the inclination direction of the displacement direction angle 8; that is, the ultrasound scanning direction, and a display showing a value of the displacement direction angle θ (as an example, 30°). The message 1002 is formed by a text prompting a change of the transmission angle of the ultrasound transmitted from the ultrasound probe 12. No particular limitations are imposed on the guide 1001 and the message 1002, so long as visible information including a text, a figure, a sign, or the like is displayed. For example, the guide and message may be formed as an arbitrary combination of the text, the figure, and the sign, or as only the text, only the figure, only the sign, etc.

Here, in the present embodiment, unlike the above-described sixth preferred embodiment, the automatic change of the transmission angle of the ultrasound (ultrasound scanning direction) transmitted from the ultrasound probe 12 is not expected. Because of this, the contents of the guide 1001 and the message 1002 are such that change of the transmission angle of the ultrasound (ultrasound scanning direction) transmitted from the ultrasound probe 12 is prompted to the user. By checking such guide 1001 and message 1002, the user can immediately understand and handle the necessity for improvement in the process with respect to the ultrasound scanning.

In the present embodiment, if the transmission angle of the ultrasound (ultrasound scanning direction) transmitted from the ultrasound probe 12 is to be automatically changed in a manner similar to that of the above-described sixth preferred embodiment, a guide and a message indicating that such change of the transmission angle (ultrasound scanning direction) has been automatically executed may be displayed on the image display 26.

### Eighth Preferred Embodiment

An ultrasound diagnostic apparatus according to an eighth preferred embodiment of the present invention will now be described with reference to the drawings. Unless otherwise particularly stated, structures are similar to those of the ultrasound diagnostic apparatus according to the first preferred embodiment.

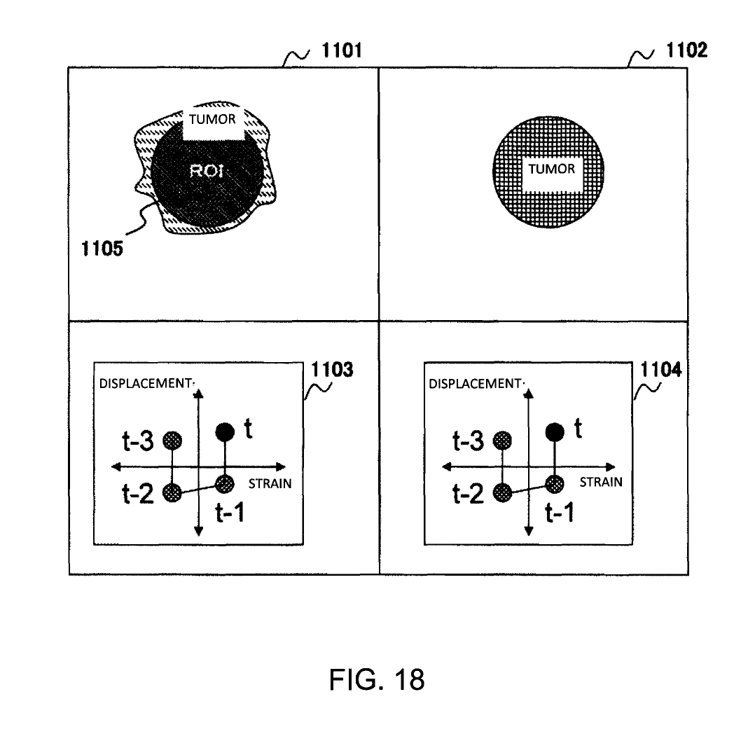

In the present embodiment, a displacement-strain coordinate is displayed as a trajectory of the parameter acquisition region along with the elasticity image and the tomographic image on the image display 26 (FIG. 1). FIG. 18 is a diagram exemplifying a displaying of an image on the image display 26 in the present embodiment. In this case, the trajectory forming unit 50 forms trajectories (displacement-strain coordinates) 1103 and 1104 showing a relationship between displacement and strain in the two-dimensional directions in the parameter acquisition region, and causes the trajectories 1103 and 1104 to be displayed on the image display 26. The trajectories 1103 and 1104 are displayed on the image display 26 along with a tomographic image 1102 and an elasticity image 1101. FIG. 18 shows an example in which the trajectories 1103 and 1104 in the parameter acquisition region are displayed along with the tomographic image 1102 and the elasticity image 1101 in a tumor site.

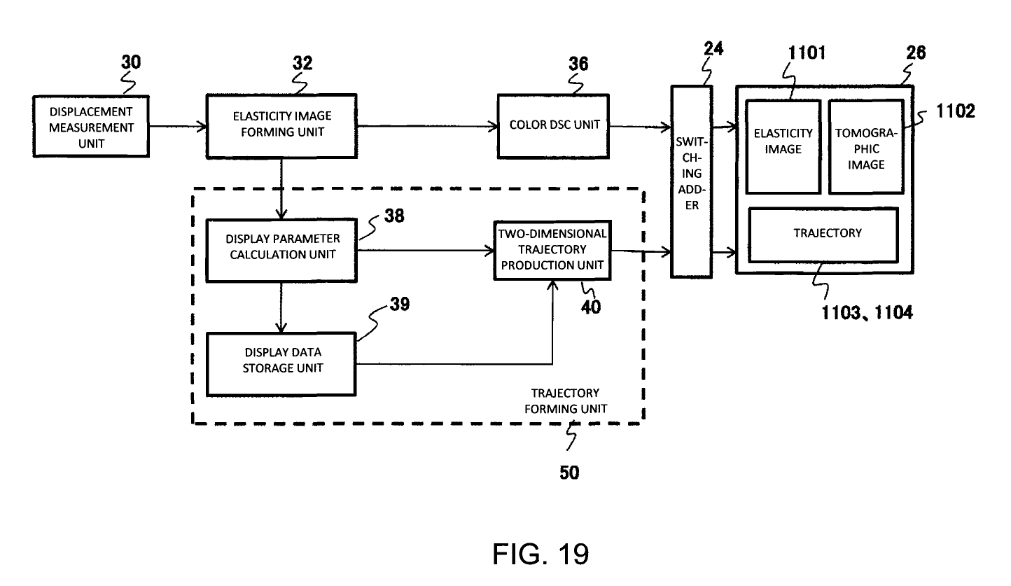

FIG. 19 is a block diagram exemplifying a structure of the trajectory forming unit 50 of the present embodiment. A difference from the block diagram (FIG. 2) of the first preferred embodiment lies in that, in addition to the trajectory forming unit 50 receiving the two-dimensional displacement distribution of the movement vector from the displacement measurement unit 30, data of the strain of the parameter acquisition region is received from the elasticity image forming unit 32. In the present embodiment, the display parameter calculation unit 38 of the trajectory forming unit 50 calculates a parameter related to the two-dimensional displacement distribution (displacement distribution with respect to the X direction and the Y direction) of the movement vector determined by the displacement measurement unit 30 and the strain calculated by the elasticity image forming unit 32.

The two-dimensional displacement distribution of the movement vector and the strain are displacement (direction and magnitude) and strain in the living body tissue corresponding to the points of the tomographic image 1102. In this case, the display parameter calculation unit 38 calculates, with regard to the two-dimensional displacement distribution of the movement vector and the strain, a parameter indicating the relationship between the displacement of the movement vector in the X direction and the strain of the parameter acquisition region (hereinafter referred to an “X direction parameter”), and a parameter indicating the relationship between the displacement of the movement vector in the Y direction and the strain of the parameter acquisition region (hereinafter referred to as a “Y direction parameter”).

The display data storage unit 39 time sequentially stores and holds the X direction parameter and the Y direction parameter calculated by the display parameter calculation unit 38.

The two-dimensional trajectory production unit 40 forms a two-dimensional trajectory based on the X direction parameter held in the display data storage unit 39 and forms a two-dimensional trajectory based on the Y direction parameter, and causes the trajectories to be displayed on the image display 26 through the switching adder 24. Alternatively, the two-dimensional trajectory production unit 40 may form the trajectory based on the X direction parameter and the Y direction parameter calculated by the display parameter calculation unit 38 in addition to or in place of the X direction parameter and the Y direction parameter held in the display data storage unit 39. With such a configuration, for example, it becomes possible to update the trajectory in real time based on the most recent X direction parameter and Y direction parameter.

In the present embodiment, the two-dimensional trajectory production unit 40 forms the trajectory (X direction displacement-strain coordinate) 1103 by time sequentially plotting the X direction parameter with the displacement with respect to the X direction and the strain as two coordinate axes (displacement axis and strain axis). Similarly, the two-dimensional trajectory production unit 40 forms the trajectory (Y direction displacement-strain coordinate) 1104 by time sequentially plotting the Y direction parameter with the displacement in the Y direction and strain as two coordinate axes (displacement axis and strain axis). The trajectories 1103 and 1104 are formed for an ROI 1105 which is the same parameter acquisition region. In this case, the ROI 1105 is set for the tumor site of the elasticity image 1101.

Alternatively, the ROI may be set for a site near the tumor site (for example, a fat site). The setting of the ROI 1105 can be achieved, for example, by the user designating a desired region in the elasticity image 1101 displayed on the image display 26 using the operation device of the interface unit 42.

In the present embodiment, the ROI 1105 is set for the elasticity image 1101, but alternatively, the ROI 1105 may be set for the tomographic image 1102 or for both the elasticity image 1101 and the tomographic image 1102. In other words, a plurality of parameter acquisition regions (ROIs) may be set.

The trajectory 1103 shown in FIG. 18 is formed by plotting the X direction parameters in the parameter acquisition region in the present and in the past on the two-dimensional coordinate axes (displacement axis and strain axis). The trajectory 1104 shown in FIG. 18 is formed by plotting the Y direction parameters in the parameter acquisition region in the present and in the past on the two-dimensional coordinate axes (displacement axis and strain axis).

In this process, the number of plots of the parameter is not particularly limited, and may be arbitrarily set, for example, according to the frame rate or the like for forming the tomographic image 1102 and the elasticity image 1101.

As an example, FIG. 18 shows trajectories 1103 and 1104 formed by plotting the X direction parameter and the Y direction parameter in the parameter acquisition region at four points in time. In the trajectories 1103 and 1104, the current point in time is set as time t, and three points in time in the past from the time t are set as time t-1, time t-2, and time t-3, in that order. The time interval between these points in time may be set to the same interval, or, alternatively, be set to be different from each other.

In the trajectories 1103 and 1104, the plotted point (parameters) of the points in time is linked by a straight line with an immediately near plotted point. Alternatively, the plotted points may be connected, for example, by an arrow line from the immediately near plotted point toward the next plotted point rather than the straight line, in order to allow understanding of the change with respect to time of the trajectories 1103 and 1104. In the trajectories 1103 and 1104, the plotted point of the current time t is displayed with a darker color than the past times t-1˜t-3, and a display showing which time the plotted point corresponds is also displayed. The display form of the plotted point is not limited to such a configuration, and, for example, the plotted points for the current time t and the past times t-1˜t-3 may be displayed with different color phases, different sizes, or the like.

In the present embodiment, by observing the trajectories 1103 and 1104, the relationship between the displacement and the strain in the parameter acquisition region can be time sequentially understood. In the living body tissue, basically, the displacement and the strain are in a proportionality relationship. However, for example, in the observation of the liver tissue during ascites, there may be cases where the displacement and the strain are not in the proportionality relationship. In a normal liver tissue, a large displacement and a large strain due to the heartbeat may be expected.

In other words, a normal liver tissue is displaced while being strained (displacement by compression). On the contrary, in hepatocirrhosis tissue, a large displacement and a small strain can be expected. That is, the hepatocirrhosis tissue is displaced without being strained (displacement by translation).

Therefore, by forming the trajectory time sequentially showing the relationship between the displacement and strain while setting the liver tissue as the parameter acquisition region, it becomes possible to judge whether the liver tissue is displaced by compression or by translation. With such a configuration, it becomes possible to judge whether the liver tissue is normal or abnormal. In other words, when the tomographic image and the elasticity image are displayed along with the trajectory, it is possible to judge whether or not these images are worth observing. Thus, the trajectory becomes useful information for judging the merits of observation for the tomographic image and the elasticity image.

As described, according to the first through eighth preferred embodiments of the present invention, a trajectory (two-dimensional displacement coordinates, displacement histogram, displacement-strain coordinate) related to the displacement in the two-dimensional directions in an arbitrary region (parameter acquisition region) of the subject 10 can be formed, and efficiency of diagnosis using the ultrasound image (elasticity image, tomographic image, or the like) in the ultrasound diagnostic apparatus can be improved.

The present invention is not limited to the above-described preferred embodiments, and various changes and modifications are possible within the scope described in the claims.

An ultrasound diagnostic apparatus according to one aspect of the present invention comprises an image forming unit that forms an ultrasound image of a diagnosis site of a subject through an ultrasound probe, an image display that displays the ultrasound image, and a trajectory forming unit that forms, based on a displacement distribution in two-dimensional directions in an arbitrary region of the ultrasound image, a trajectory related to a displacement of the region, and that causes the trajectory to be displayed on the image display.

According to such a structure, the trajectory of displacement in two-dimensional directions in an arbitrary region of the ultrasound image can be formed and displayed. By observing the trajectory, the displacement in the ultrasound image provided for diagnosis can be tracked in a wide range. In addition, by observing the trajectory, the image precision of the ultrasound image can be judged, and the image precision can thus be improved.

As a result, for example, a mammary gland, a liver, or the like for which the displacement in a wide range in the two-dimensional directions must be tracked can be accurately diagnosed.

In an ultrasound diagnostic apparatus according to another aspect of the present invention, the trajectory forming unit time sequentially calculates a parameter related to the displacement of the region based on the displacement distribution in the two-dimensional directions, and forms the trajectory on coordinate axes based on the calculated parameter.

According to such a structure, a parameter at an arbitrary point in time related to the displacement of the region can be selected, the trajectory can be formed, and the trajectory can be understood on the coordinate axis.

In an ultrasound diagnostic apparatus according to another aspect of the present invention, the trajectory forming unit calculates the parameter related to the displacement in the two-dimensional directions in the region based on the displacement distribution in the two-dimensional directions, and forms the trajectory by plotting the parameter in the present and in the past on the coordinate axes in the two-dimensional directions.

According to such a structure, by observing the trajectory, the change with respect to time of the displacement in the two-dimensional directions in the region from the past to the present can be understood on the coordinate axes.

In an ultrasound diagnostic apparatus according to another aspect of the present invention, the trajectory forming unit calculates a parameter showing a relationship between a magnitude and a frequency of the displacement in the two-dimensional directions in the region based on the displacement distribution in the two-dimensional directions, and forms the relationship between the magnitude and frequency of the displacement as the trajectory based on the parameter in the present and in the past.

According to such a structure, by observing the trajectory, the relationship between the magnitude and frequency of the displacement in the two-dimensional directions in the region from the past to the present can be understood.

In an ultrasound diagnostic apparatus according to another aspect of the present invention, the trajectory forming unit calculates a parameter showing a relationship between a displacement and a strain in the two-dimensional directions in the region based on the displacement distribution in the two-dimensional directions, and forms the trajectory by plotting the parameter in the present and in the past on coordinate axes of the displacement and the strain.

According to such a structure, by observing the trajectory, the relationship between the displacement and strain of the region from the past to the present can be understood. In this manner, for example, even for a living body tissue in which the displacement and the strain are not in the proportionality relationship, it becomes possible to judge whether the living body tissue is normal or abnormal.

In an ultrasound diagnostic apparatus according to another aspect of the present invention, the trajectory forming unit calculates the parameter as a statistical value including at least one of an average, a variance, a maximum, a minimum, a center value, and a frequency of the displacement of the region based on the displacement distribution in the two-dimensional directions.

According to such a structure, a tendency of the displacement of the region can be statistically tracked, and errors in the parameter can be effectively removed. With the use of such a parameter, a more appropriate trajectory can be formed.

In an ultrasound diagnostic apparatus according to another aspect of the present invention, the trajectory forming unit forms the trajectory including an appropriate displacement range in the two-dimensional directions in the region, and causes the trajectory including the appropriate displacement range to be displayed on the image display.

According to such a structure, by observing the trajectory, it is possible to easily understand whether or not the displacement of the region is appropriately tracked. As a result, the image precision of the ultrasound image provided for the diagnosis can be accurately judged.

In an ultrasound diagnostic apparatus according to another aspect of the present invention, the trajectory forming unit removes a trajectory which does not fall within the appropriate displacement range, selects only a trajectory falling within the appropriate displacement range, and causes the trajectory to be displayed on the image display.

According to such a structure, it is possible to display only a trajectory falling within the appropriate displacement range, and a trajectory that does not fall within the appropriate displacement range does not need to be observed. Therefore, the work for the user to select a trajectory useful for diagnosis and an ultrasound image synchronized with the trajectory can be omitted.

In an ultrasound diagnostic apparatus according to another aspect of the present invention, the trajectory forming unit calculates a displacement direction of the region from the trajectory related to the displacement of the region, and changes a transmission direction of an ultrasound transmitted from the ultrasound probe to the subject based on the displacement direction.

According to such a structure, the transmission direction of ultrasound can be automatically made to coincide with the displacement direction of the region. As a result, a trajectory in which the displacement direction is biased along the transmission direction of the ultrasound can be formed.

In an ultrasound diagnostic apparatus according to another aspect of the present invention, the trajectory forming unit calculates a displacement direction of the region from the trajectory related to the displacement of the region, and causes visible information including at least one of a text, a figure, and a sign related to the displacement direction to be displayed on the image display.

According to such a structure, information related to the displacement direction of the region can be notified to the user. With this process, for example, the user can understand and handle necessity or the like for improvement of the process for the ultrasound scanning.

In an ultrasound diagnostic apparatus according to another aspect of the present invention, the image forming unit comprises a tomographic image forming unit that forms a tomographic image as the ultrasound image based on ultrasound tomographic data of the diagnosis site, and causes the tomographic image to be displayed on the image display, and an elasticity image forming unit that determines a strain or a modulus of elasticity of a tissue in the diagnosis site based on the ultrasound tomographic data, that forms an elasticity image in the diagnosis site as the ultrasound image based on the determined strain or modulus of elasticity, and that causes the elasticity image to be displayed on the image display, and the trajectory forming unit causes the trajectory to be displayed on the image display along with at least one of the tomographic image and the elasticity image.

According to such a structure, along with the tomographic image and the elasticity image in the diagnosis site, a trajectory of displacement in the two-dimensional directions in an arbitrary region of these images can be formed and displayed. Therefore, by observing the trajectory along with the tomographic image and the elasticity image, the image precisions for the tomographic image and the elasticity image can be judged, and the image precisions can be improved.

In an ultrasound diagnostic apparatus according to another aspect of the present invention, the elasticity image forming unit forms a displacement image in the diagnosis site as the ultrasound image based on a vector indicating a direction and a magnitude of the displacement in the two-dimensional directions of points in the tomographic image, and causes the displacement image to be displayed on the image display.

According to such a structure, the trajectory of the displacement in the two-dimensional directions in the region can be formed and displayed while checking the vector display in the displacement image. As a result, the precision of the trajectory can be improved.

In an ultrasound diagnostic apparatus according to another aspect of the present invention, the trajectory forming unit forms the trajectory related to the displacement of the region in the two-dimensional directions based on the displacement distribution in the two-dimensional directions in at least one region which is set for at least one image of the ultrasound image.

According to such a structure, the region can be freely set for any of the tomographic image, the elasticity image, and the displacement image at the diagnosis site, and the trajectory of the displacement of the region can be formed.

In an ultrasound diagnostic apparatus according to another aspect of the present invention, the trajectory forming unit forms, based on displacement distributions in the two-dimensional directions in a plurality of the regions which are set for at least one image of the ultrasound image, trajectories related to the displacements of the plurality of regions in the two-dimensional directions, on the same coordinate axes or different coordinate axes.

According to such a structure, a plurality of the regions can be set for any of the tomographic image, the elasticity image, and the displacement image, and the trajectories of the displacements of the plurality of the regions can be formed. Therefore, by simultaneously displaying these trajectories, the plurality of trajectories can be observed while comparing with each other.

### EXPLANATION OF REFERENCE NUMERALS

10 SUBJECT; 12 ULTRASOUND PROBE; 14 TRANSMITTING UNIT; 16 RECEIVING UNIT; 17 ULTRASOUND TRANSMISSION/RECEPTION CONTROLLER; 18 PHASING ADDER; 20 TOMOGRAPHIC IMAGE FORMING UNIT; 22 BLACK-AND-WHITE DSC; 24 SWITCHING ADDER; 26 IMAGE DISPLAY; 28 RF FRAME DATA SELECTION UNIT; 30 DISPLACEMENT MEASUREMENT UNIT; 32 ELASTICITY IMAGE FORMING UNIT; 36 COLOR DSC; 38 DISPLAY PARAMETER CALCULATION UNIT; 39 DISPLAY DATA STORAGE UNIT; 40 TWO-DIMENSIONAL TRAJECTORY PRODUCTION UNIT; 42 INTERFACE UNIT; 44 CONTROLLER; 46 PRESSURE MEASUREMENT UNIT; 50 TRAJECTORY FORMING UNIT.

1. An ultrasound diagnostic apparatus, comprising:
an image forming unit that forms an ultrasound image of a diagnosis site of a subject through an ultrasound probe;
an image display that displays the ultrasound image; and
a trajectory forming unit that forms, based on a displacement distribution in two-dimensional directions in an arbitrary region of the ultrasound image, a trajectory related to a displacement of the region, and that causes the trajectory to be displayed on the image display.

2. The ultrasound diagnostic apparatus according to claim 1, wherein
the trajectory forming unit time sequentially calculates a parameter related to the displacement of the region based on the displacement distribution in the two-dimensional directions, and forms the trajectory on coordinate axes based on the calculated parameter.

3. The ultrasound diagnostic apparatus according to claim 2, wherein
the trajectory forming unit calculates the parameter related to the displacement in the two-dimensional directions in the region based on the displacement distribution in the two-dimensional directions, and forms the trajectory by plotting the parameter in the present and in the past on the coordinate axes in the two-dimensional directions.

4. The ultrasound diagnostic apparatus according to claim 2, wherein
the trajectory forming unit calculates a parameter showing a relationship between a magnitude and a frequency of the displacement in the two-dimensional directions in the region based on the displacement distribution in the two-dimensional directions, and forms the relationship between the magnitude and the frequency of the displacement as the trajectory based on the parameter in the present and in the past.

5. The ultrasound diagnostic apparatus according to claim 2, wherein
the trajectory forming unit calculates a parameter showing a relationship between a displacement and a strain in the two-dimensional directions in the region based on the displacement distribution in the two-dimensional directions, and forms the trajectory by plotting the parameter in the present and in the past on coordinate axes of the displacement and the strain.

6. The ultrasound diagnostic apparatus according to claim 2, wherein
the trajectory forming unit calculates the parameter as a statistical value including at least one of an average, a variance, a maximum, a minimum, a center value, and a frequency of the displacement of the region based on the displacement distribution in the two-dimensional directions.

7. The ultrasound diagnostic apparatus according to claim 1, wherein
the trajectory forming unit forms the trajectory including an appropriate displacement range in the two-dimensional directions in the region, and causes the trajectory including the appropriate displacement range to be displayed on the image display.

8. The ultrasound diagnostic apparatus according to claim 7, wherein
the trajectory forming unit removes a trajectory which does not fall within the appropriate displacement range, selects only a trajectory falling within the appropriate displacement range, and causes the trajectory to be displayed on the image display.

9. The ultrasound diagnostic apparatus according to claim 1, wherein
the trajectory forming unit calculates a displacement direction of the region from the trajectory related to the displacement of the region, and changes a transmission direction of an ultrasound transmitted from the ultrasound probe to the subject based on the displacement direction.

10. The ultrasound diagnostic apparatus according to claim 1, wherein
the trajectory forming unit calculates a displacement direction of the region from the trajectory related to the displacement of the region, and causes visible information including at least one of a text, a figure, and a sign related to the displacement direction to be displayed on the image display.

11. The ultrasound diagnostic apparatus according to claim 1, wherein
the image forming unit comprises:
a tomographic image forming unit that forms a tomographic image as the ultrasound image based on ultrasound tomographic data of the diagnosis site, and causes the tomographic image to be displayed on the image display; and
an elasticity image forming unit that determines a strain or a modulus of elasticity of a tissue in the diagnosis site based on the ultrasound tomographic data, that forms an elasticity image in the diagnosis site as the ultrasound image based on the determined strain or modulus of elasticity, and that causes the elasticity image to be displayed on the image display, and
the trajectory forming unit causes the trajectory to be displayed on the image display along with at least one of the tomographic image and the elasticity image.

12. The ultrasound diagnostic apparatus according to claim 11, wherein
the elasticity image forming unit forms a displacement image in the diagnosis site as the ultrasound image based on a vector indicating a direction and a magnitude of the displacement in the two-dimensional directions of points in the elasticity image, and causes the displacement image to be displayed on the image display.

13. The ultrasound diagnostic apparatus according to claim 11, wherein
the trajectory forming unit forms the trajectory related to the displacement of the region in the two-dimensional directions based on the displacement distribution in the two-dimensional directions in at least one region which is set for at least one image of the ultrasound image.

14. The ultrasound diagnostic apparatus according to claim 13, wherein
the trajectory forming unit forms, based on displacement distributions in the two-dimensional directions in a plurality of the regions which are set for at least one image of the ultrasound image, trajectories related to the displacements of the plurality of regions in the two-dimensional directions, on same coordinate axes or different coordinate axes.

15. A method of displaying a trajectory, comprising the steps of:
forming an ultrasound image of a diagnosis site of a subject through an ultrasound probe;
forming, based on a displacement distribution in two-dimensional directions in an arbitrary region of the ultrasound image, a trajectory related to a displacement of the region; and
displaying the ultrasound image and the trajectory.
